(19) United States
(12) Patent Application Publication
(10) Pub. No.: US 2012/0310312 A1
(43) Pub. Date: Dec. 6, 2012

(54) Precision-Controlled Cooling System for Inducing Diving Reflex and Achieving Safe Hypothermic Central Nervous System Protection

(76) Inventor: Thomas Chih-Han Yee, Las Vegas, NV (US)

(21) Appl. No.: 13/151,354

(22) Filed: Jun. 2, 2011

(51) Int. Cl. A61F 7/12; A61F 7/10

(52) U.S. Cl. 607/105; 607/104

(57) ABSTRACT

A cooling system for inducing diving reflex and central nervous system protective hypothermia has a cooler, a refrigerant dispenser, a cooling assembly, a controller and an infusing unit. The refrigerant dispenser connects the cooler. The cooling assembly connects the refrigerant dispenser and communicates with the cooler and has a head cooling set. The controller coordinates the refrigerant dispenser and the infusing unit according to the detection of the physical signal including naso-pharyngeal temperature, and cerebral oximetry signal. The infusing unit connects to the controller and has a catheter. When in use, the cooled liquid is pumped into (1) head cooling set covering the face of the patient to induce diving reflex, (2) the infusing unit having an esophago-gastric tube to lower the temperature inside the stomach. Therefore, an effective, precise diving reflex and central nervous system protective hypothermia can be achieved by this invention.

### BACKGROUND OF THE INVENTION

1. Field of the Invention

The present invention relates to a cooling system for inducing and maintaining diving reflex and protection of central nervous system of human patients who have suffered anoxic brain injury, spinal injury or trauma to central nervous system.

2. Description of the Prior Arts

Diving reflex is a physiological phenomenon of mammals, including humans, wherein as the face is exposed to icy cold fluid, the heart rate slows, metabolism slows, and the body preferentially directs oxygenated blood to the brain and heart, preserving the viability of these key organs. This phenomenon has been repeatedly demonstrated in incidents of successful resuscitation of cold water drowning victims, who survived longer periods of lifelessness than warm water drowning victims.

As known by the applicant, none of current techniques provide integrated device for inducing diving reflex and maintaining diving reflex and protection of central nervous system of human patients who have suffered anoxic brain injury, spinal injury or trauma to central nervous system. Therefore, there is an urgent need for an effective, economic and convenient device for inducing diving reflex in a patient.

### SUMMARY OF THE INVENTION

The main objective of the invention is to provide a cooling system for inducing diving reflex and effective cooling of the central nervous system, by using a cooling assembly that would cool a liquid to as low a temperature as 2 degrees Celsius and an infusing pump unit to circulate iced-cold saline to immerse the patient's head, face, neck, torso and pump ice-cold saline into the stomach of the patient for obtaining an efficient heat exchange rate and effectively lowering the temperature of the patient to achieve central nervous system protection.

The cooling system for inducing diving reflex and central nervous hypothermic protection in accordance with the present invention has a cooler, a refrigerant dispenser, a cooling assembly, a controller and an infusing unit.

The cooler cools and supplies a liquid. The refrigerant dispenser connects the cooler and pumps the liquid from and/or back to the cooler. The cooling assembly connects the refrigerant dispenser and communicates with the cooler and has a head cooling set. The head cooling set has a bag made of heat-conducting material. The bag has a chamber, an inlet and an outlet. The inlet and the outlet connect to the refrigerant dispenser. The chamber accommodates the liquid from the refrigerant dispenser through the inlet. The controller connects to the refrigerant dispenser and detects both the nasopharyngeal temperature and the cerebral oximetry data from a patient. The infusing unit connects to the controller and has a refrigerator, catheter and a flow control unit. The refrigerator connects to the handling module of the controller and supplies cold saline. The catheter has a tip, an afferent lumen and an efferent lumen. The afferent lumen has an end and an opening. The end of the afferent lumen is formed at the tip of the catheter. The opening is formed at the end of the afferent lumen. The efferent lumen has an end, a throughhole and is 1 inch in diameter. The end of the efferent lumen is also formed at the tip of the catheter. The throughhole is formed at a distance from the end of the efferent lumen. The flow control unit connects the catheter and the refrigerator pumps ice-cold saline from or back to the refrigerator.

When in use, the head cooling set is mounted around a subject patient and the catheter can be inserted and transfer ice-cold saline into the head unit to lower the temperature of the patient. The controller coordinates the refrigerant dispenser and the infusing unit according to the feedback signals of the nasopharyngeal temperature probe and the cerebral oximetry probe from the forehead. Therefore, an effective diving reflex can be induced by the cooling system to desired brain temperature as reflected by the nasopharyngeal temperature probe and by the cerebral oximetry probe on the forehead without endangering the patient.

This invention takes advantage of the diving reflex, producing superior hypothermic protection of the patient's brain and spinal cord, by not only cooling the brain, neck, back, torso, stomach, esophagus, aorta, and heart, but also sharply cooling the patient's face to induce the mammalian “diving reflex.” This present invention goes significantly beyond the scope of the current art of cooling patients for medical reasons. The current art of achieving cooling of patients does not provide for the induction of diving reflex.

Nor does the current art provide simultaneous three-prong approach of this invention: (1) Cooling the head, neck, and torso externally with controlled icy-cold water, (2) Cooling the face with controlled icy-cold water to induce the diving reflex, (3) Cooling the stomach internally through an Esophageal-Gastric tube circuit, effectively cooling the neighboring internal organs of heart, aorta, vena cava and liver. Finally, the current art does not control the cooling by monitoring the brain temperature (via nasal pharyngeal temperature probe), or by monitoring the cerebral oximetry (real-time, non-invasive, continuous monitoring of brain oxygenation and perfusion) while this invention includes such precision control of the cooling process to optimize induction of diving reflex and to achieve desired central nervous system protection.

Other objectives, advantages and novel features of the invention will become more apparent from the following detailed description when taken in conjunction with the accompanying drawings.

### BRIEF DESCRIPTION OF THE DRAWINGS

FIG. 1 an operational top view of one portion of a cooling system for inducing diving reflex in accordance with the present invention;

FIG. 2A is an operational top view of another portion of the cooling system for inducing diving reflex in FIG. 1;

FIG. 2B is an enlarged side view of the catheter in FIG. 2A;

FIG. 3 is a side view of the head cooling set in accordance with the present invention;

FIG. 4 is a perspective view of the holder in accordance with the present invention;

FIG. 5 is an operational side view of the combination of the head cooling set and the holder in FIGS. 3 and 4;

FIG. 6 is a side view of the torso cooling set in accordance with the present invention; and

FIG. 7 is a scheme illustrating connections among the handling module, the detector, and the input unit of the controller, the cooler, and the refrigerant dispenser in accordance with the present invention.

### DETAILED DESCRIPTION OF THE PREFERRED EMBODIMENTS

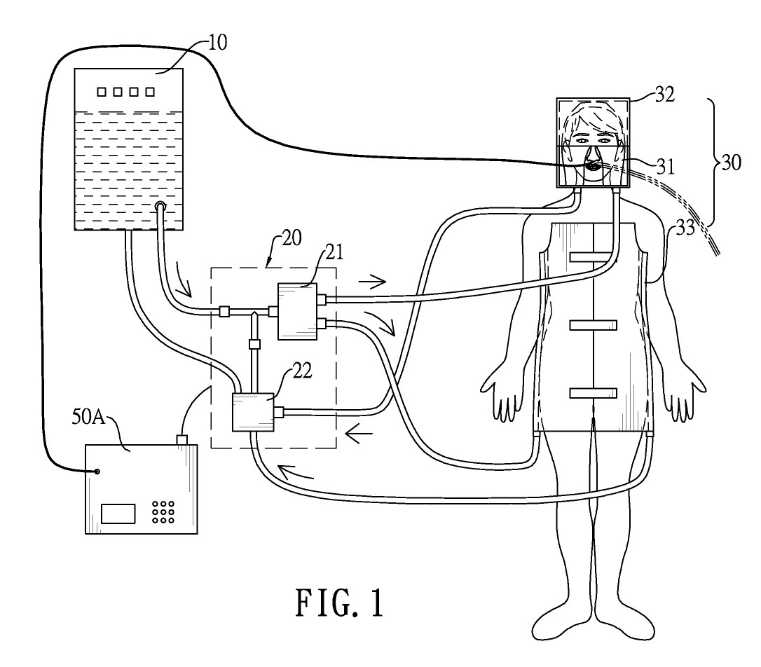

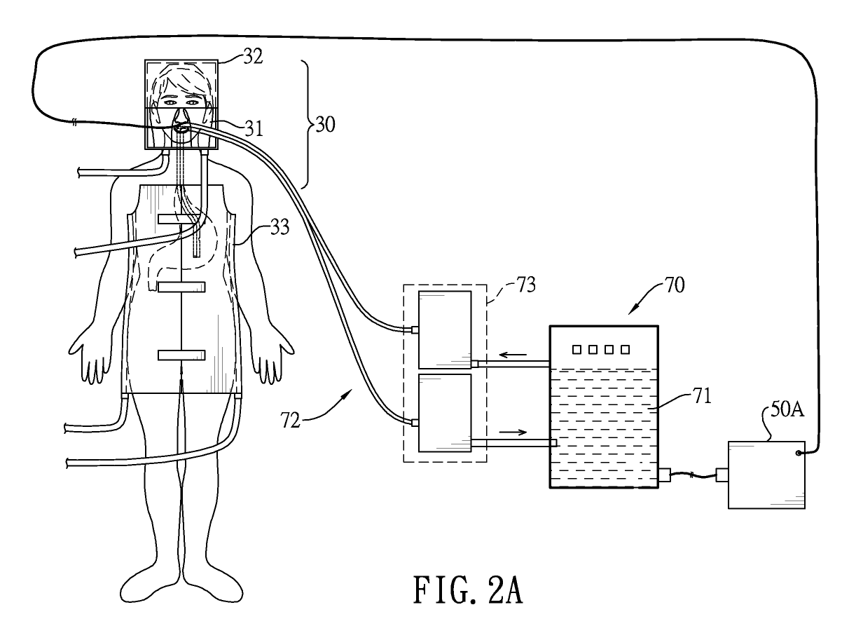

As shown in FIGS. 1 and 2A, a cooling system for inducing diving reflex in accordance with the present invention has a cooler (10), a refrigerant dispenser (20), a cooling assembly (30), a controller (50A) and an infusing unit (70).

The cooler (10) cools and supplies a liquid, and has a cooling tank. The cooling tank accommodates 15 gallons of water and cools it to 2° C.

The refrigerant dispenser (20) connects to the cooler (10) and pumps the liquid from and/or back to the cooler (10). The refrigerant dispenser (20) has at least one pump (21, 22).

The cooling assembly (30) connects to the refrigerant dispenser (20) and communicates with the cooler (10) through the refrigerant dispenser (20), which forms a loop. The loop is regulated by the refrigerant dispenser (20). The cooling assembly (30) has a head cooling set, a holder (32) and a torso cooling set.

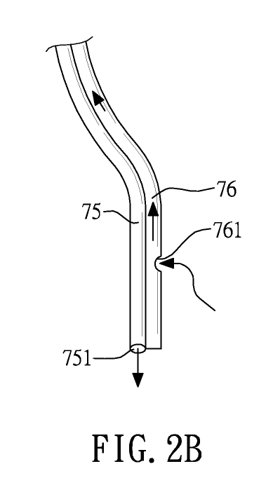

As shown in FIG. 3, the head cooling set connects to the refrigerant dispenser (20) and has a bag (31) and at least one tube (34, 35). The bag (31) is transparent, made of transparent plastic materials, is of a size of 100 cm by 100 cm and has a chamber for accommodating the liquid. The volume of the chamber is about 20 liters. The bag (31) has an inlet (311) and an outlet (312). The inlet (311) and the outlet (312) connect to the at least one pump (21, 22) of the refrigerant dispenser (20). Each tube (34, 35) is flexible, mounted in the chamber of the bag (31), is of a diameter of 1 inch and has an open end, a closed end and multiple orifices. The open end of each tube (34, 35) connects to the inlet (311) or the outlet (312). The orifices of the at least one tube (34, 35) communicate the chamber of the bag (31) with the inlet (311) and/or the outlet (312) through the at least one tube (34, 35).

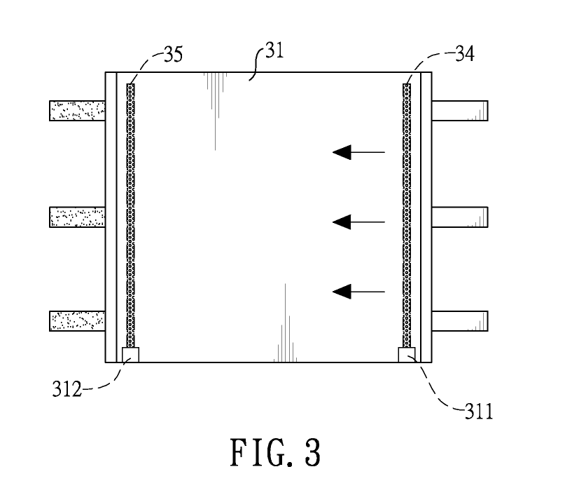

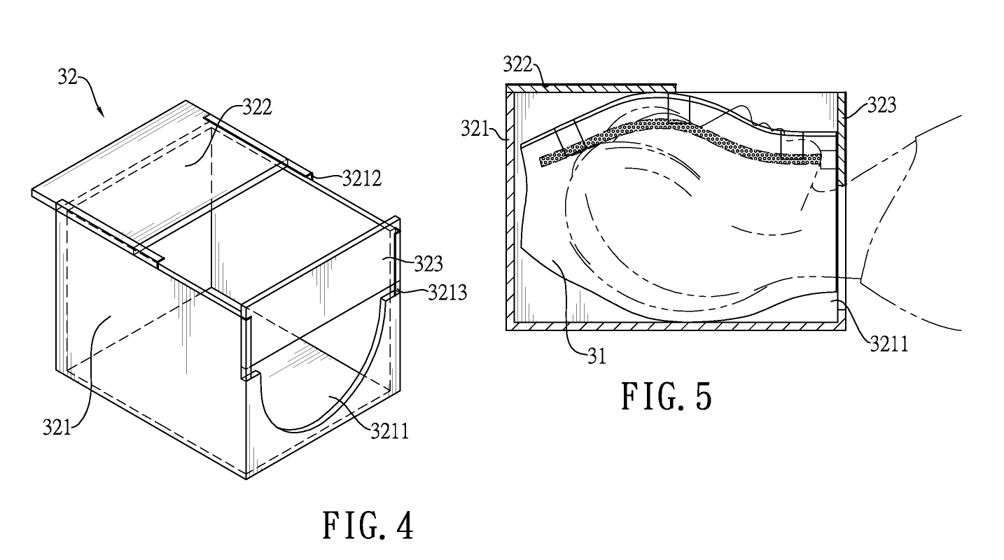

With reference to FIGS. 4 and FIG. 5, the holder (32) is transparent, is made of transparent plastic materials (such as acrylic), holds the bag to shape the bag to cover the patient's face, head and neck with the bag and has a base (321), a first cover (322) and a second cover (323). The base (321) has a top, a front side, a rear side, an opening (3211), a cavity, a top track (3212) and a side track (3213). The opening (3211) is formed at the front side of the base (321) and communicates with the cavity. The top track (3212) is formed on the top of the base (321) close to the rear side and has two parallel grooves. The first cover (322) is movably mounted on the top track (3212) and has two edges. The two edges of the first cover (322) are respectively mounted in the grooves of the top track (3212). The side track (3213) is formed on the front side of the base (321) and has two parallel grooves. The second cover (323) is movably mounted on the side track (3213) and has two edges. The two edges of the second cover (323) are respectively mounted in the grooves of the side track (3213).

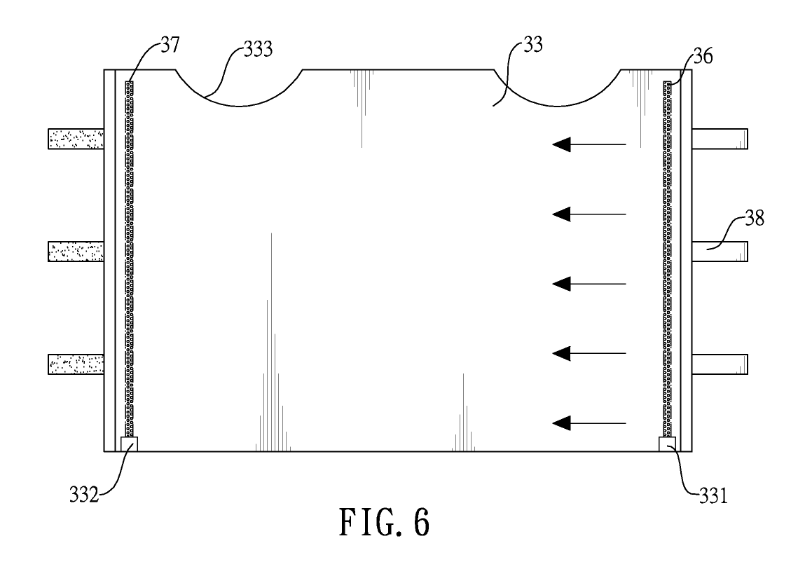

As shown in FIG. 6, the torso cooling set connects to the refrigerant dispenser (20) and has a pouch (33), at least one pipe (36, 37) and multiple fasteners (38). The pouch (33) is transparent, is plastic material, is of a size of 120 cm by 120 cm, and has a chamber for accommodating the liquid. The volume of the chamber is about 30 liters. The pouch (33) has an inlet (331), an outlet (332) and two arched gaps (333). The inlet (331) and the outlet (332) connect to the at least one pump (21, 22) of the refrigerant dispenser (20). The two arched gaps (333) are located at a side of the pouch (33). Each pipe (36, 37) is flexible and mounted in the chamber of the pouch (33) adjacent to another side of the pouch (33), is of a diameter of 1 inch and has an open end, a closed end and multiple orifices. The open end of each pipe (36, 37) connects to the inlet (331) or the outlet (332). The orifices of the at least one pipe (36, 37) communicate the chamber of the pouch (33) with the inlet (331) or the outlet (332) through the at least one pipe (36, 37). The fasteners (38) may be velcro pad and are attached to the pouch (33). Each two fasteners (38) detachably connect to each other in pairs for fixing the pouch (33), for being rolled up and wrapped around a subject matter.

In a preferred embodiment in accordance with the present invention, the bag (31) of the head cooling set and the pouch (33) of the torso cooling set is made of material selected from the group consisting of: polyethylene (PE), polyvinyl chloride (PVC), chlorinated polyethylene (CPE), polyamide (PA), polyethylene terephthalate (PET) and combination thereof.

As shown in FIG. 8, the controller (50A) connects to the cooler (10), the refrigerant dispenser (20) and the infusing unit (70) and has a detector (40), a handling module (50) and an input unit (60).

The detector (40) detects a physical signal (eq. nasopharyngeal temperature and cerebral oximetry data) from the patient, transforms the physical signal into a secondary signal and transfers the secondary signal. In a preferred embodiment, the physical signal can be, but is not limited to: cerebral temperature, body core temperature, blood oxygen content and cerebral oxygen content. In a preferred embodiment, the detector (40) includes, but is not limited to: cerebral thermometer, core body thermometer, pulse oximeter and cerebral oximeter. In another preferred embodiment, the core body thermometer may be infrared body thermometer. In still another preferred embodiment, the detector (40) includes a nasal sensor for detecting nasal pharyngeal temperature.

The handling module (50) receives the secondary signal from the detector (40) and trigger the refrigerant dispenser (2) in accordance with the secondary signal, whereby the liquid in the cooler (10) is pumped into the bag (31) of the head cooling set and/or the pouch (33) of the torso cooling set and then pumped back to the cooler (10) by the refrigerant dispenser (20). In a preferred embodiment, the handling module (50) connects to the cooler (10) to control temperature of the liquid to be lowered to a predetermined temperature. In another preferred embodiment, the handling module (50) is programmed to have a default parameter of temperature as a reference to the secondary signal and the physical signal.

The input unit (60) connects to the handling module (50) for setting the default parameter of the handling module (50).

With further reference to FIGS. 2A and 2B, the infusing unit (70) connects to the handling module (50) of the controller (50A) and has a refrigerator (71), a catheter (72) and a flow control unit (73). The refrigerator (71) connects to the handling module (50) of the controller (50A) and supplies cold saline. The catheter (72) has a tip, an afferent lumen (75) and an efferent lumen (76). The afferent lumen (75) has an end, an opening (751) and a diameter of 1 cm. The end of the afferent lumen (75) is formed at the tip of the catheter (72). The opening (751) is formed at the end of the afferent lumen (75). The efferent lumen (76) has an end, a throughhole (761) and a diameter of 1 cm. The end of the efferent lumen (76) is also formed at the tip of the catheter (72). The throughhole (761) is formed at a distance of 15 cm from the end of the efferent lumen (76). The flow control unit (73) connects the catheter (72) and the refrigerator (71) and has at least one pump (74). The pump (74) pumps the ice-cold saline from or back to the refrigerator (71).

When in use, with reference to FIGS. 1, 2A and 5, the bag (31) of the head cooling set is wrapped around the head of a patient and allows its nose and mouth to remain exposed for airway management. The base (321) of the holder (32) is then mounted around the head of the patient wrapped in the bag (31) to allow the head and the bag (31) to be located in the chamber of the base (321) and the neck of the patient to extend through the opening (3211) of the base (321). The first cover (322) and the second cover (323) are respectively mounted on the top track (3212) on the top and the side track (3213) at the front side over the opening (3211) of the base (321) to hold the head of the patient wrapped in the bag (31) steady. The pouch (33) is then wrapped around the torso of the mammal to a state allowing the arms of the mammal to extend through the arched gap (333) of the pouch (33) and to stay fixed by the fasteners (38).

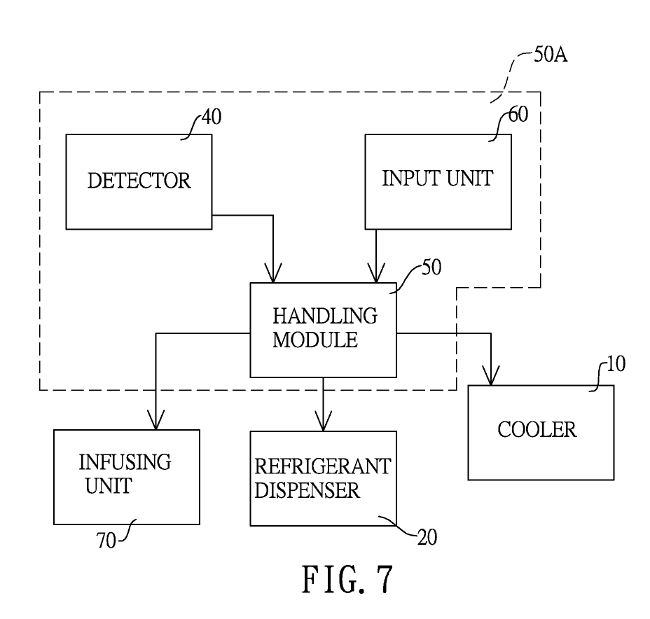

With further reference to FIG. 7, the refrigerant dispenser (20) pumps the cold liquid at 2 to 4° C. in the cooler (10) into the bag (31) through the inlet (311) and/or the tube (34, 35) and into the pouch (33) through the inlet (331) and/or the pipe (36, 37) in the cooling assembly (30). Since each tube (34, 35) and pipe (36,37) has a closed end, the cold liquid in the tube (34,35) and the pipe (36, 37) penetrates through the orifices to be evenly distributed over and mixed in the chamber of the bag (31) or the pouch (33). Further, the bag (31) and the pouch (33) are flexible and capable of being shaped to abut surfaces of the patient to maximize the contact area for better heat exchange, resulting in an excellent diving reflex of the patient. As shown in FIG. 2A, the catheter (72) of the infusing unit (70) is inserted into the patient's mouth and into the stomach to a distance of 50 cm past the teeth. The ice-cold saline is continuously pumped by the pumps (73, 74) into the afferent lumen (75) at a rate of 2 liters a minute through the opening (751) into the stomach, while saline in the stomach is withdrawn at the same rate through the throughhole (761) into the efferent lumen (76).

In a preferred embodiment, while the secondary signal from the detector (40) is higher than the default parameter of temperature in handling module (50), the handling module (50) triggers the refrigerant dispenser (20) to pump the cold liquid in the cooler (10) into the bag (31) of the head cooling set and/or the pouch (33) of the torso cooling set, and triggers the pumps (73, 74) of the infusing unit (70) to pump ice-cold saline into the stomach of the mammal. While the secondary signal from the detector (40) is lower than the default parameter of temperature in handling module (50), the handling module (50) triggers the refrigerant dispenser (20) to pump the liquid in the bag (31) of the head cooling set and/or the pouch (33) of the torso cooling set back to the cooler (10) or triggers the pumps (73, 74) of the infusing unit (70) to pump saline in the stomach of the mammal back to the refrigerator (71). In another preferred embodiment, the default parameter of handling module (50) is 33° C. While the secondary signal corresponds to a physical signal, such as cerebral temperature, lower than 33° C., the handling module (50) triggers the refrigerant dispenser (20) to decrease the flow rate of the cold liquid pumped into the bag (31) of the head cooling set and/or the pouch (33) of the torso cooling set or draw liquid in the bag (31) of the head cooling set and/or the pouch (33) of the torso cooling set back into the cooler (10). The handling module (50) also triggers the at least one pump (73, 74) of the infusing unit (70) to withdraw saline in the stomach of the mammal. Therefore, the temperature of the mammal can be steadily maintained by precisely inducing the diving reflex and achieving hypothermia. Based on the technical features of the cooling system for inducing diving reflex and hypothermia to protect the central nervous system as described above, an user can coordinate the cooler (10), the refrigerant dispenser (20) and the infusing unit (70) according to the detection of the detector (40) by using the controller (50A), resulting in keeping the patient at a steady low temperature so as to effectively achieve the desired clinical result in saving patient lives and central nervous system functions.

Even though numerous characteristics and advantages of the present invention have been set forth in the foregoing description, together with details of the structure and features of the invention, the disclosure is illustrative only. Changes may be made in the details, especially in matters of shape, size, and arrangement of parts within the principles of the invention to the full extent indicated by the broad general meaning of the terms in which the appended claims are expressed.

What is claimed is:

1. A cooling system for inducing diving reflex, comprising:
a cooler for cooling liquid;
a refrigerant dispenser connecting to the cooler and pumping the liquid from and/or back to the cooler;
a cooling assembly connecting to the refrigerant dispenser and communicating with the cooler and having
a head cooling set having
a bag having
a chamber for accommodating the liquid; and
an inlet and an outlet connecting to the refrigerant dispenser; and
a holder holding the bag to shape the bag to cover a patient's face, head and neck with the bag;
a controller connecting to the refrigerant dispenser, detecting a physical signal from a mammal; and
an infusing unit connecting to the controller and having
a refrigerator connecting to the controller for cooling saline;
a catheter having
a tip;
an afferent lumen having
an end formed at the tip of the catheter; and
an opening formed at the end of the afferent lumen; and
an efferent lumen having
an end formed at the tip of the catheter; and
an throughhole formed at a distance from the end of the efferent lumen; and
a flow control unit connecting to the catheter and the refrigerator to pump the saline from or back into the refrigerator.

2. The cooling system for inducing diving reflex of the claim 1, wherein the head cooling set further includes:
at least one tube mounted in the bag and having
an open end connecting to the inlet; and
multiple orifices communicating the chamber of the bag with the inlet of the bag through the tube.

3. The cooling system for inducing diving reflex of the claim 1, further comprising:
a torso cooling set connecting to the refrigerant dispenser and having
a pouch having
a chamber for accommodating liquid; and
an inlet; and an outlet connecting to the refrigerant dispenser; and
multiple fasteners attached to the pouch.

4. The cooling system for inducing diving reflex of the claim 3, further comprising: at least one pipe mounted in the chamber of the pouch and having
an open end connecting to the inlet of the pouch; and
multiple orifices communicating the chamber of the pouch with the inlet through the pipe.

5. The cooling system for inducing diving reflex of the claim 1, wherein the controller having
a detector for detecting a physical signal from a patient, transforming the physical signal into a secondary signal and transferring the secondary signal; and
a handling module receiving the secondary signal from the detector and triggering the refrigerant dispenser.

6. The cooling system for inducing diving reflex of the claim 5, wherein the handling module has a default parameter, whereby when the secondary signal from the detector is higher than the default parameter of temperature in the handling module, the handling module triggers the refrigerant dispenser to pump cold liquid in the cooler into the bag of the head cooling set and/or the pouch of the torso cooling set, and triggers the flow control unit of the infusing unit to pump ice-cold saline into the stomach of the mammal.

7. The cooling system for inducing diving reflex of the claim 5, wherein the controller has an input unit connecting to the handling module and for setting the default parameter of the handling module.

8. The cooling system for inducing diving reflex of the claim 5, wherein the detector is selected form the group consisting of: cerebral thermometer, core body thermometer, pulse oximeter and cerebral oximeter.

9. The cooling system for inducing diving reflex of the claim 5, wherein the detector includes a nasal sensor for detecting nasal pharyngeal temperature.

10. The cooling system for inducing diving reflex of the claim 3, wherein the controller having
a detector for detecting a physical signal from a mammal, transforming the physical signal into a secondary signal and transferring the secondary signal; and
a handling module receiving the secondary signal from the detector and triggering the refrigerant dispenser.

11. The cooling system for inducing diving reflex of the claim 10, wherein the handling module has a default parameter, whereby when the secondary signal from the detector is higher than the default parameter of temperature in the handling module, the handling module triggers the refrigerant dispenser to pump cold liquid in the cooler into the bag of the head cooling set and/or the pouch of the torso cooling set, and triggers the flow control unit of the infusing unit to pump ice-cold saline into the stomach of the mammal.

12. The cooling system for inducing diving reflex of the claim 10, wherein the controller has an input unit connecting to the handling module and for setting the default parameter of the handling module.

13. The cooling system for inducing diving reflex of the claim 10, wherein the detector is selected form the group consisting of: cerebral thermometer, core body thermometer, pulse oximeter and cerebral oximeter.

14. The cooling system for inducing diving reflex of the claim 10, wherein the detector includes a nasal sensor for detecting nasal pharyngeal temperature.
